(19) United States
(12) Patent Application Publication
(10) Pub. No.: US 2002/0132983 A1
(43) Pub. Date: Sep. 19, 2002

(54) Antibodies as chimeric effector cell receptors against tumor antigens

(76) Inventor: Richard P. Junghans, Boston, MA (US)

Correspondence Address: Richard P. Junghans, Ph.D., M.D., One Lyndeboro Place, Boston, MA, 02116, US

(21) Appl. No.: 10/006,773

(22) Filed: Dec. 10, 2001

Related U.S. Application Data
(60) Provisional application No. 60/250,089, filed on Nov. 30, 2000.
(60) Provisional application No. 60/250,087, filed on Nov. 30, 2000.

(51) Int. Cl. C07K 1/00; C07K 14/00; C07K 17/00

(52) U.S. Cl. 530/350

(57) ABSTRACT

This invention relates to specific antibodies against ganglioside GD3 called MB3.6 and against protein prostate specific membrane antigen (PSMA) called 3D8, 4D4 and 3E11 when prepared as chimeric molecules with signaling molecules of T cells and other effector cells, and the use thereof in the treatment of cancers expressing these antigens.

### STATEMENT ON FEDERALLY-SPONSORED R&D

No federal funds were used in the creation of this invention.

### BACKGROUND OF THE INVENTION

More than 500,000 Americans die each year from cancers that have proven refractory to traditional methods of treatment, for which new strategies are urgently required. Tumor-associated antigens are selected for therapeutic targets based on their high expression on tumor tissue and a lower expression in normal tissues that will plausibly allow selective targeting of tumorous expression of the antigen. Ganglioside GD3 is expressed at high levels on melanoma, small cell lung cancer and other neuroendocrine tumors. Prostate-specific membrane antigen (PSMA) is selectively expressed at high levels in prostate cancer and other tumors. T cells can penetrate virtually every biologic space and have the power to dispose of normal or malignant cells as seen in viral and autoimmune diseases and in the rare spontaneous remissions of cancer. However, T cells are readily tolerant to self or tumor antigens, and “immune surveillance” has manifestly failed in every cancer that is clinically apparent. There is a strong need and value for means to direct T cells against GD3-expressing and PSMA-expressing cancers. This patent describes specific molecules and means to achieve this goal.

### BRIEF SUMMARY OF THE INVENTION

An antibody against GD3 has been prepared called MB3.6. GD3 is expressed at high levels on melanoma and other neuroendocrine tumors, and low levels on normal tissues. Antibodies against PSMA have been prepared called 3D8, 4D4, 3E11. PSMA is expressed principally in prostate tissue, a non-essential organ, and in prostate cancers. It is the goal of this patent to supply the specificities and affinities to patient T cells without regard for their “endogenous” T cell receptor repertoire, directed by antibody-defined recognition to kill malignant cells based on their expression of antigen. This is achieved by preparing chimeric molecules of these specific antibodies with molecules derived from T cells or related effector cell molecules, which will redirect T cells or other effector cells against the tumor cells in a focused anti-tumor immune response by “re-educating” the patient's immune system.

### BRIEF DESCRIPTION OF DRAWINGS

FIG. 1 shows a chimeric antibody-T cell receptor that employs the zeta chain of the TCR. In this example, a single chain Fv (sFv) version of hMN14 is linked by a CD8α hinge to the TCR zeta chain. The CD8α hinge has been further modified to remove the cysteines involved in CD8 dimerization to improve surface expression.

FIG. 2A shows the near absence of heterodimer molecules when the native CD8α hinge is employed, although it would be predicted to be the dominant species, with a lower net expression of chimeric molecule relative to endogenous zeta chain. FIG. 2B shows the effect of removing the cysteines, which now allows much increased net expression of chimeric molecule when heterodimer can be expressed.

FIG. 3 shows diagram and DNA sequence of a chimeric sFv IgTCR, including the CD8α hinge modified-to-remove cysteines, within a retroviral vector. This example IgTCR molecule (using hMN14 antibody specific to CEA antigen, not part of this application) occupies nucleotides 2426 to 3766. (The vector sequences are incidental.) Equivalent versions using the antibodies MB3.6, 3D8, 4D4, 3E11 are prepared in analogous manner to create IgTCR, or other Ig-chimeric molecules.

FIG. 4 shows the DNA sequence of:

A., B. leader plus VH and leader plus VL that specifies MB3.6.

C. As example, the VL and leader are joined with linker to VH to create MB3.6 sFv as shown, that is subsequently used in creating chimeric molecules. Other means of generating sFv are possible and included under this claim, as well as other means of creating antibody chimeric molecules under the intent of this invention.

D., E. leader plus VH and leader plus VL that specifies 3D8 (includes C domain sequences).

F., G. leader plus VH and leader plus VL that specifies 4D4 (includes C domain sequences).

H., I. leader plus VH and leader plus VL that specifies 3E11 (includes C domain sequences).

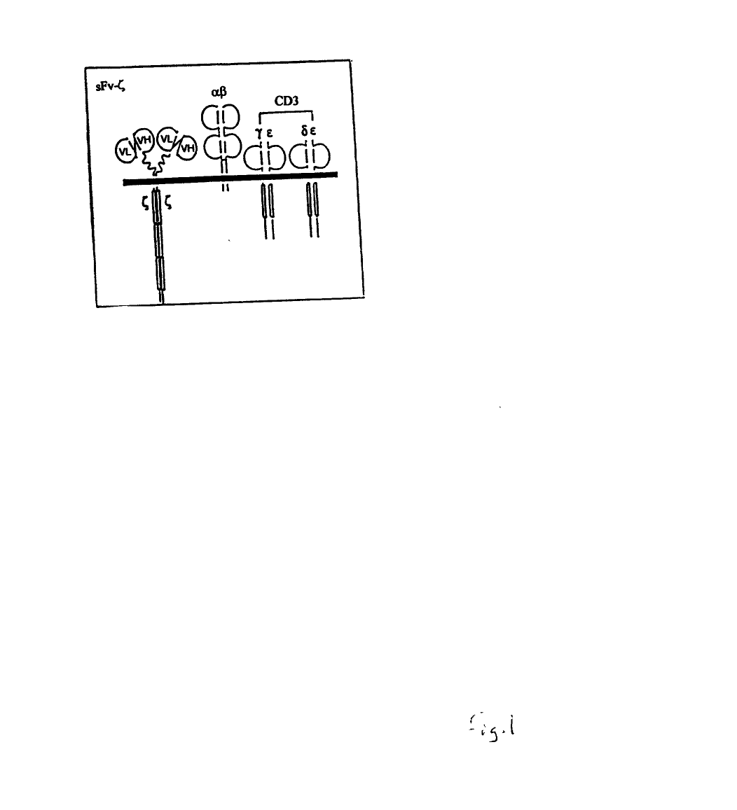

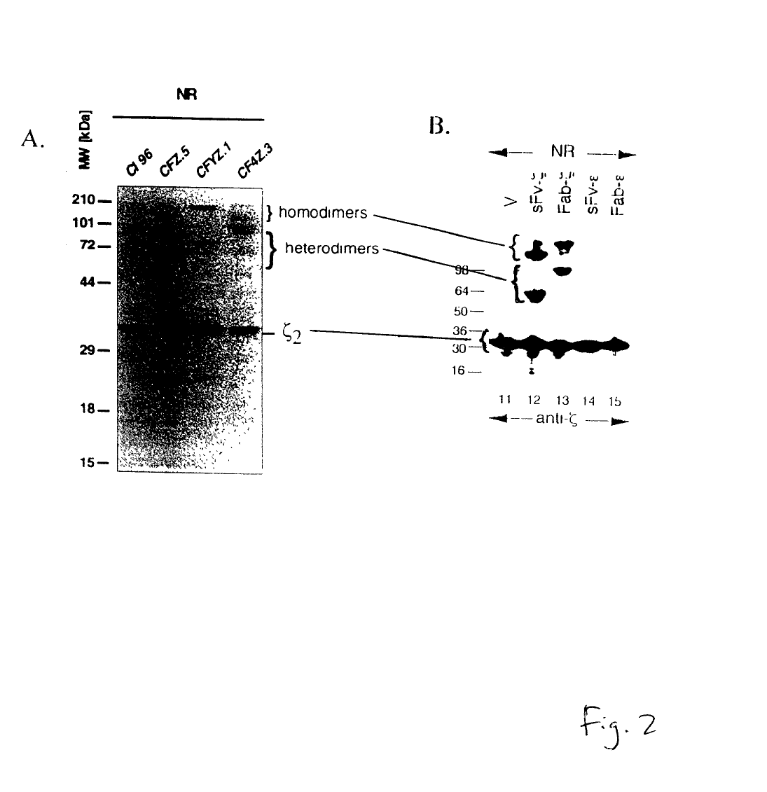

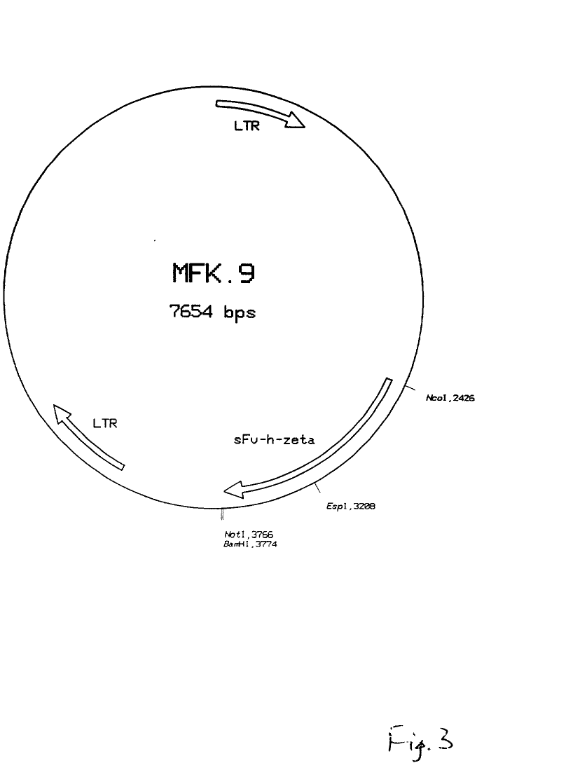

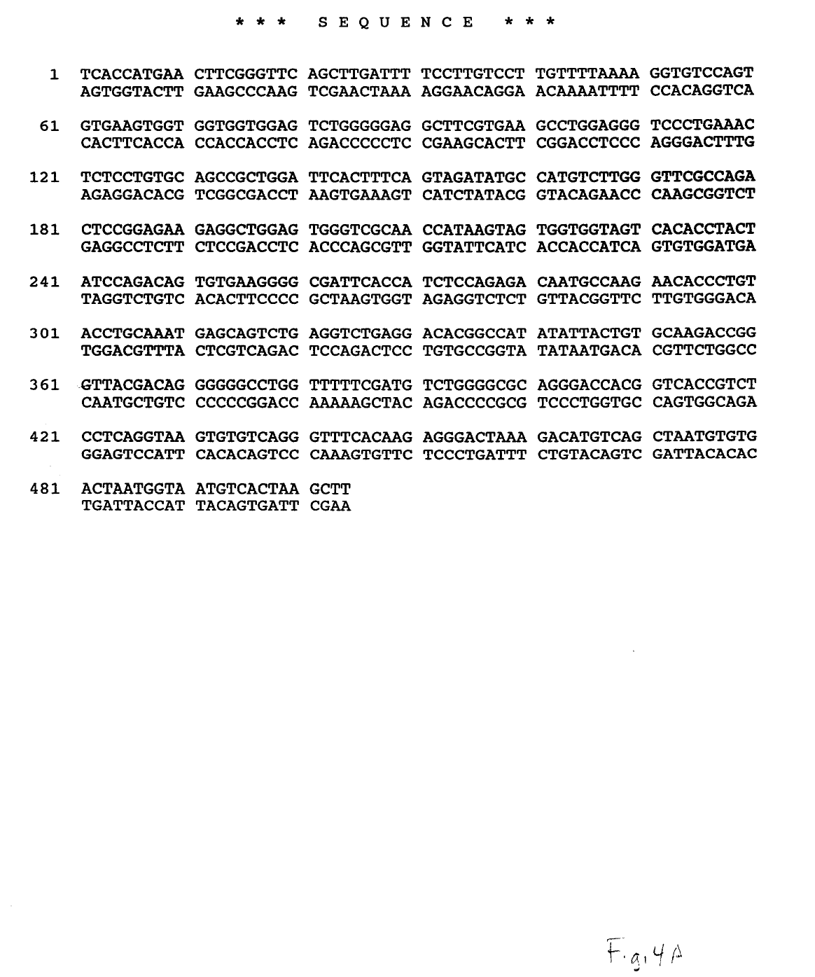

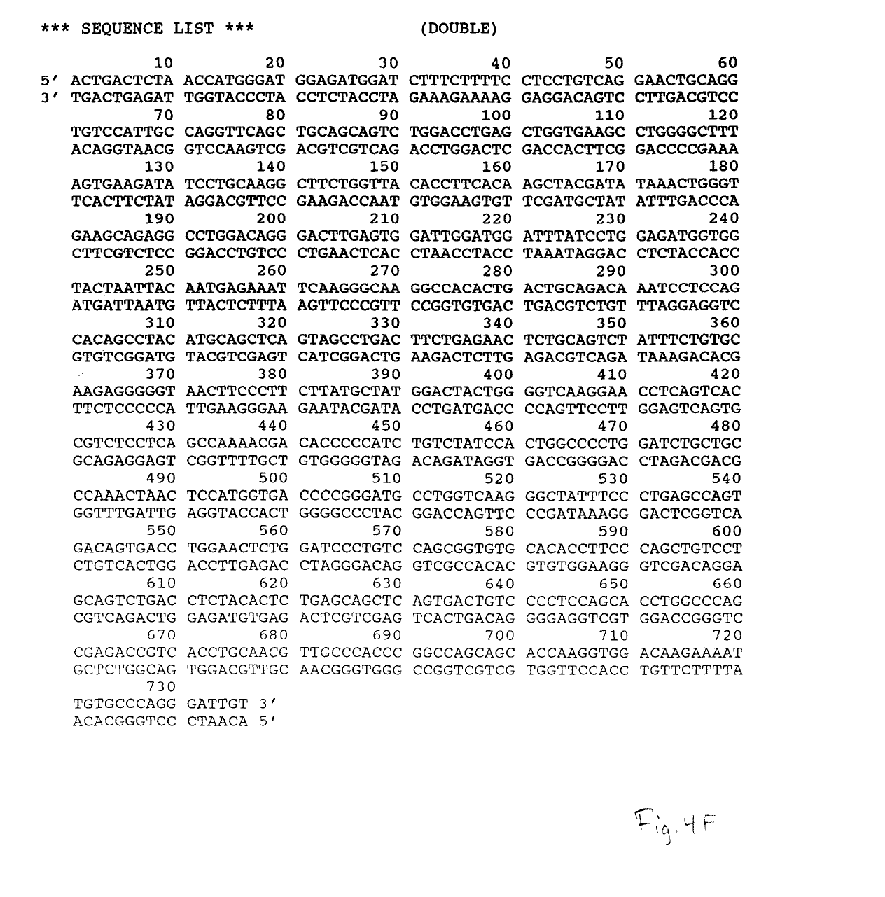

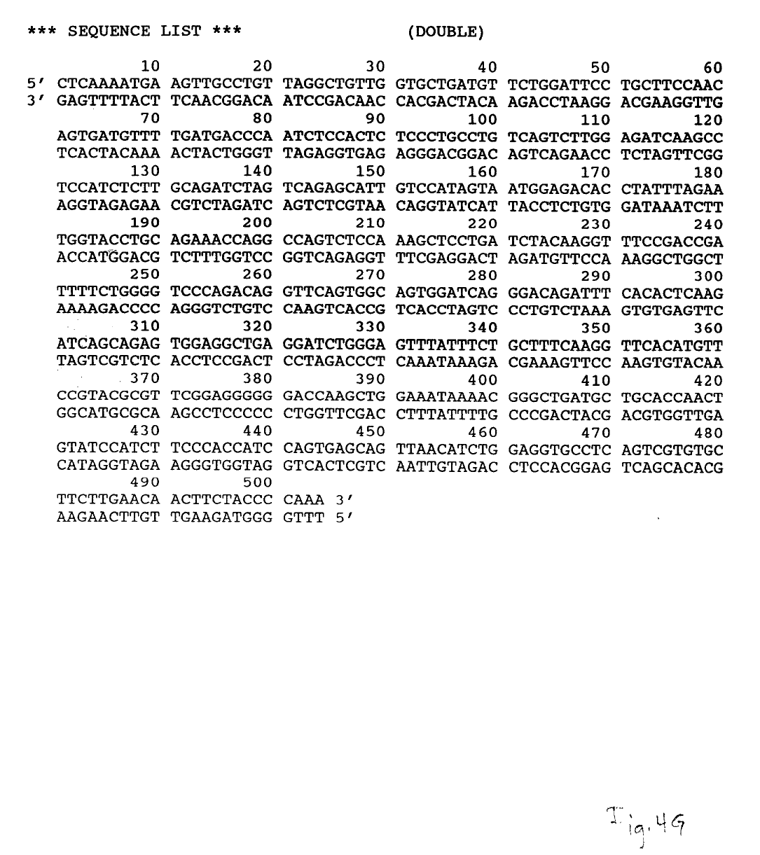

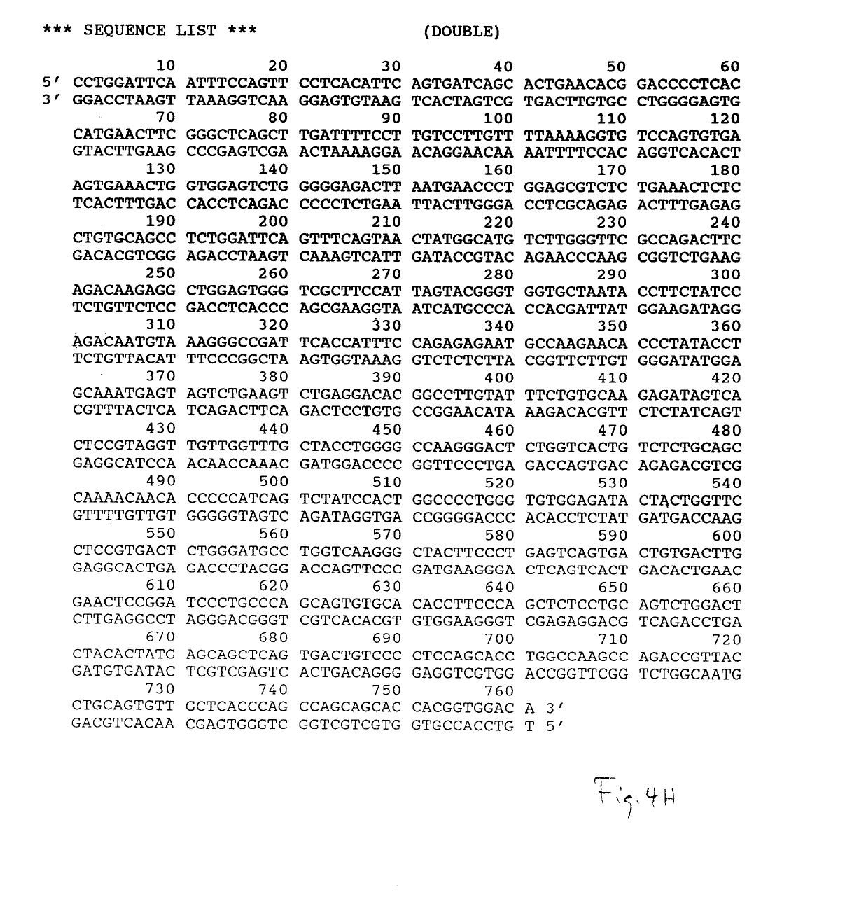

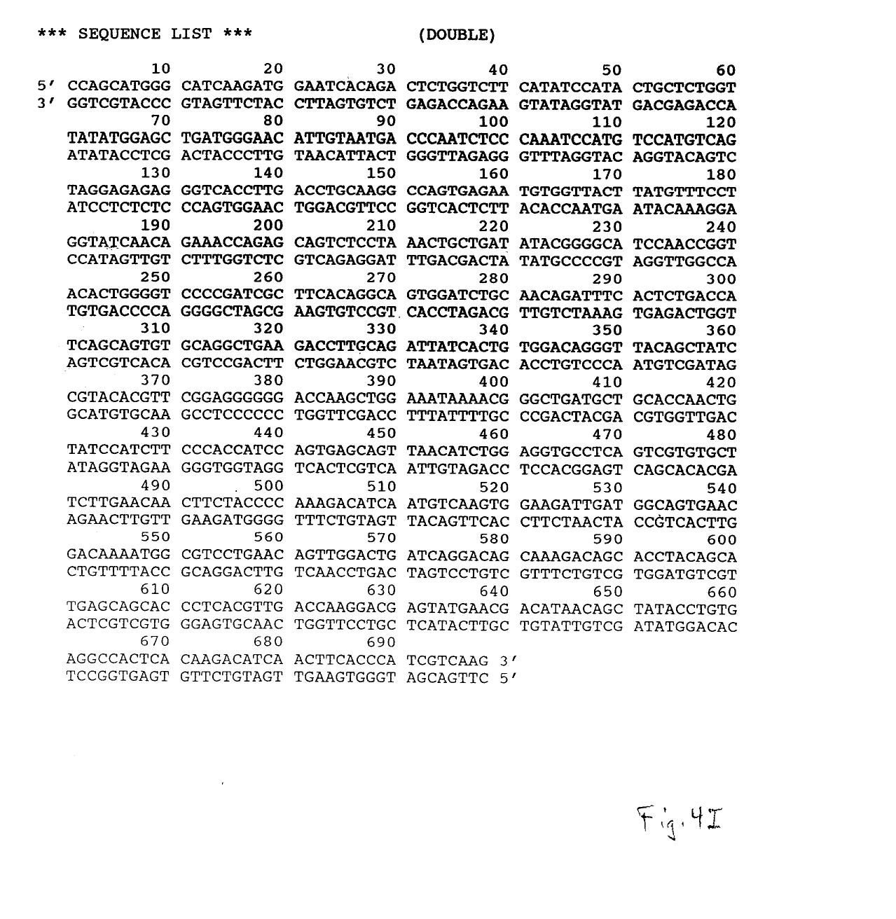

These sequences are modified to prepare the sFv used in FIG. 1 and FIG. 3, and similarly for other constructs.

FIG. 5 shows example of the effect of MB3.6 IgTCR-modified T cells in killing GD3-positive tumor cells, but sparing GD3-negative cells. Other examples with 3D8, 4D4, 3E11 would show specific killing against PSMA-positive cells but not against PSMA-negative cells.

FIG. 6 shows example of the effect of IgTCR (signal 1) using hMN14 antibody chimerics against CEA (not part of this application) on causing sustained CEA+ tumor cell killing when stimulated in conjunction with CD28 (signal 2) stimulation of the gene-modified T cells via B7 antigen expressed in the tumor cells. (A) Signal 1 alone from tumor cells leads to AICD with declining effector cell numbers, that is reversed with signal 1+2. (B) Signal 1 leads to limited duration of tumor killing because of declining T cell numbers. (C) Signal 1+2 leads to sustained tumor killing because of the sustained and expanding T cell numbers. This example justifies design of IgCD28 molecules to modify patient T cells to supply the second signal on contact with antigen that is necessary to suppress effector cell death and achieve sustained killing activity. The intent of this example is the expectation of utility with analogous constructs using the Ig sequences of this application.

FIG. 7 shows an example of a design for an IgCD28 using MB3.6, 3D8, 4D4, 3E11. This also uses a modified CD8α hinge. Similar designs for other chimeric molecules with these antibodies are envisioned, with or without hinge that is the same or different.

### DETAILED DESCRIPTION OF THE INVENTION

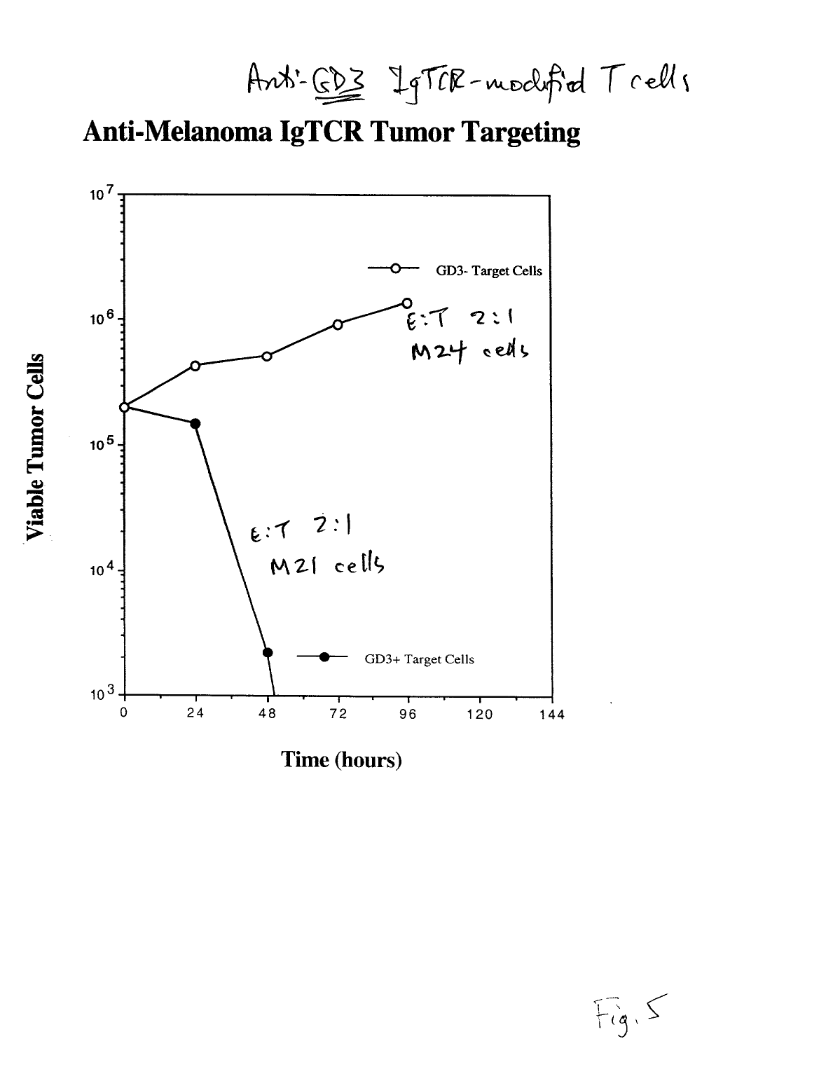

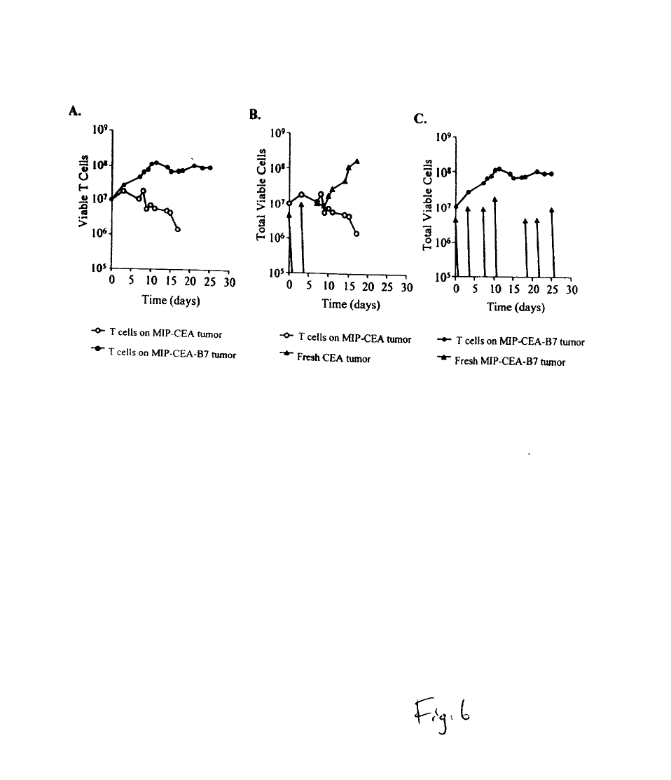

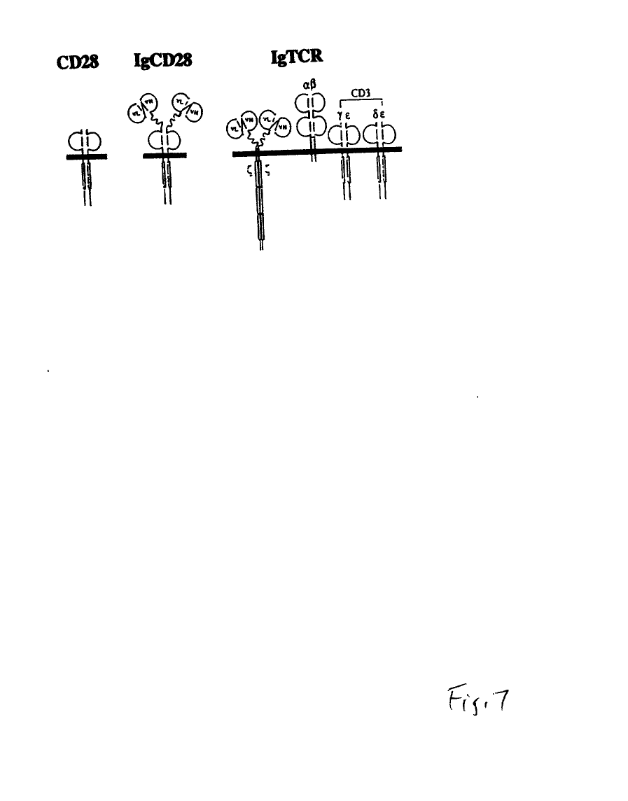

This patent is intended to cover all chimeric molecules created with the specified antibodies (Ig) (MB3.6, 3D8, 4D4, 3E11) (defined by the variable region sequences of FIG. 4) or their derivatives with cell surface molecules which could be used in redirecting and/or activating T cells or other effector cells in the recognition and attack against tumors expressing the antigens recognized by these antibodies. Other specific antibodies which the inventor or his agents obtain with rights will be similarly appended as claims at such future appropriate time. The chimeric molecules of this claim include, but are not limited to, the following molecules: IgTCR (FIGS. 1&3), which has an antibody binding domain from these antibodies fused to one or more chains of the T cell receptor complex; IgCD28 (FIG. 7), which has an antibody binding domain from these antibodies fused to the CD28 T cell co-receptor molecule; IgLFA-1, which has an antibody binding domain from these antibodies fused to the LFA-1 T cell co-receptor/adhesion molecule; IgCD2, which has an antibody binding domain from these specific antibodies fused to the CD2 T cell co-receptor/adhesion molecule; and by analogy, any other T cell or effector cell molecules which are usefully employed in chimeric structures with these antibody binding domains. The chimeric molecules may themselves incorporate cytoplasmic signaling domains, as in the previous examples. Or the chimeric molecules may instead be non-signaling, such as examples of Ig linked to TCR α or β chains, or Ig linked to Fc receptor (FcR) non-signaling chains, that in turn associate with signaling chains to activate cellular functions. These chimeric molecules may additionally incorporate spacer domains or epitope tags. Single-chain Fv (sFv) versions of these antibodies have been favored for use in these constructs, but Fab or other IgG chimeric molecules would be equally included under this invention. The initial description of some of these preparations is contained in Yun et al, 2000. This demonstrates reduction to practice of the concepts contained herein for the MB3.6 antibody, with expectation of similar results for the other antibodies specified in this invention.

The invention additionally allows for the presence of a (GSGGS)3 linker in the sFv of the Ig portion of the chimeric molecules (e.g., FIG. 4C). Whereas the sFv antibodies may frequently not fold properly to maintain stability, I included the extra serine to improve hydration and sFv folding versus the typical (GGGGS)3 linker that has been associated in some cases with abolished or diminished sFv affinity (e.g., Brinkmann et al, 1993). This strategy with an antibody not covered under this patent (hMN14) led to an sFv virtually indistinguishable from the monovalent binding affinity of the parental antibody (Nolan et al, 1999). Such tests have not been performed with the current antibodies, but all have maintained antigen recognition after sFv modification with this linker.

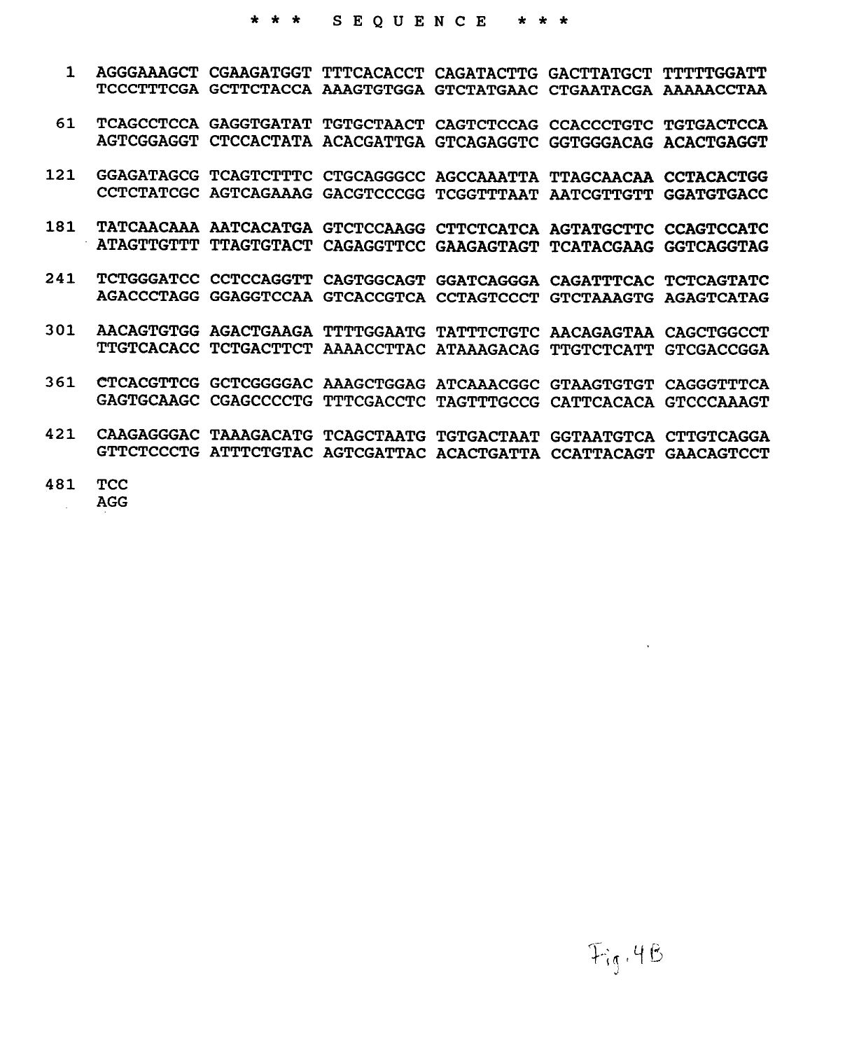

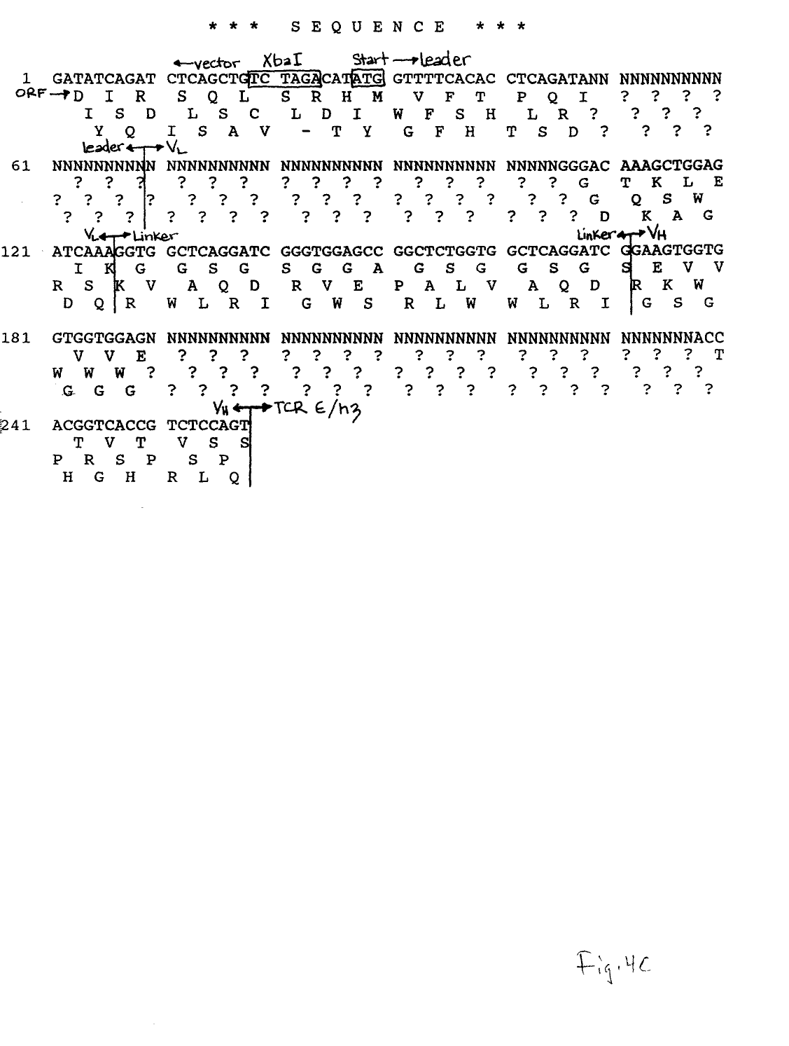

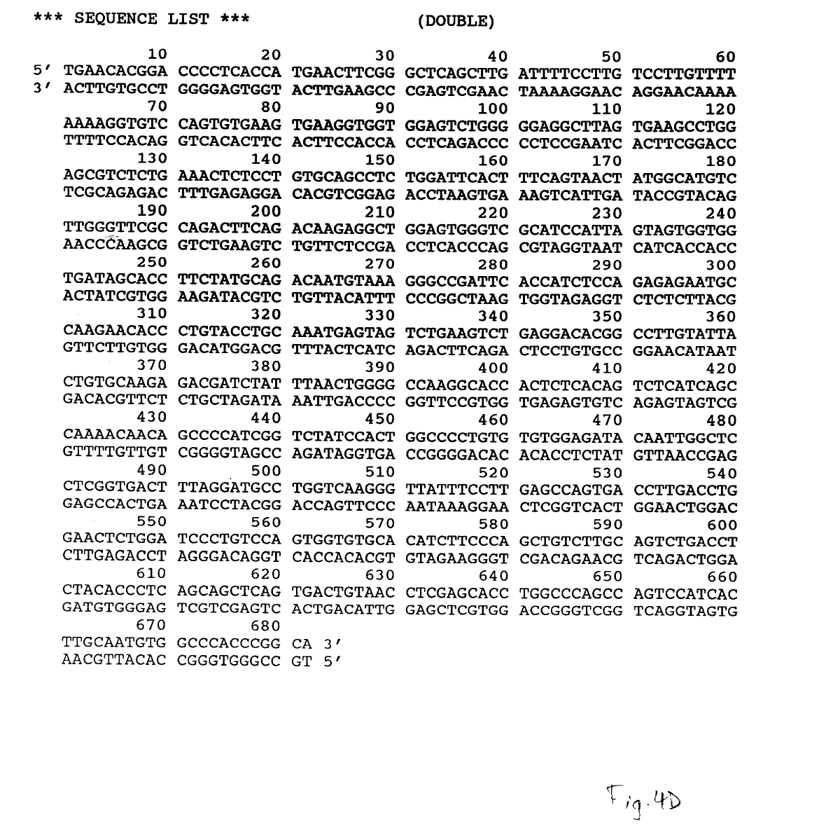

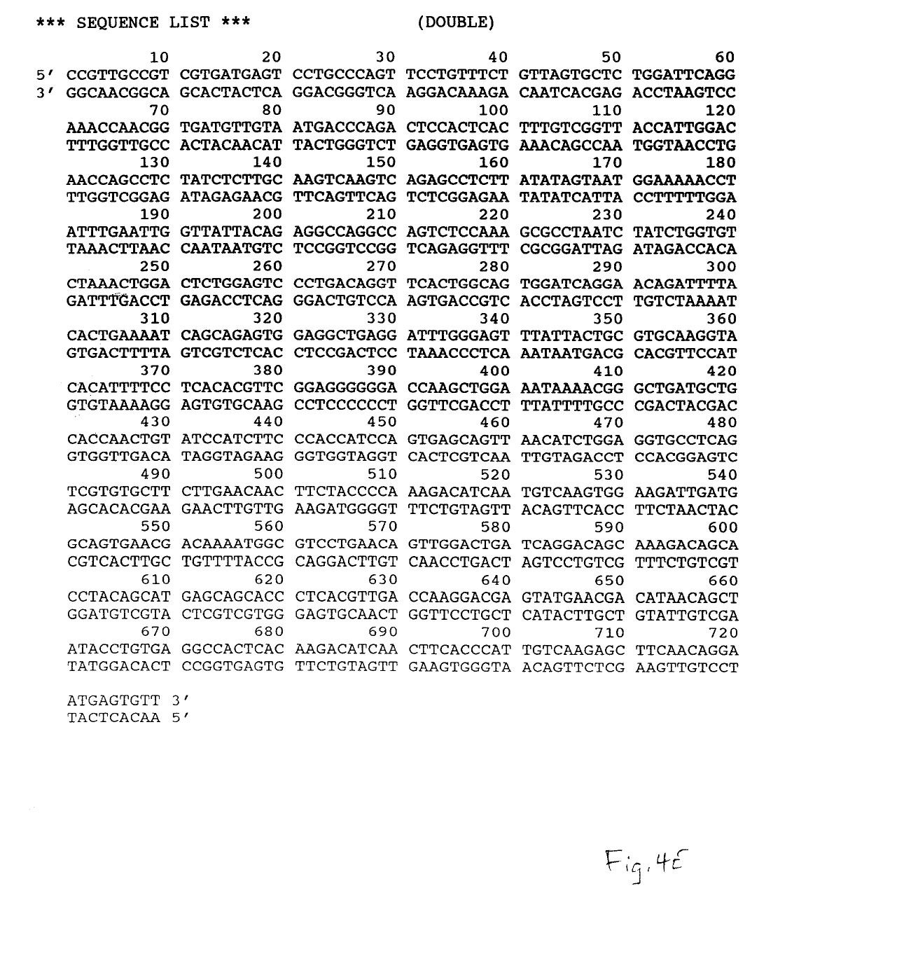

The invention additionally allows for the modification-to-remove cysteines in the CD8α hinge domain to improve the surface expression of the chimeric molecules (Nolan et al, 1999). Free cysteines of the hinge of the heterodimer of zeta:sFv-hinge-zeta target this molecular complex for destruction, reducing the net amount of chimeric molecule expression on the cell surface. (The homodimer (sFv-hinge-zeta)₂ has safe pairing of cysteines to spare this specific configuration from destruction. More heterodimer is expected because of binomial considerations where the endogenous zeta exceeds the transduced zeta chimera as is typical.) This principle is demonstrated by the poor expression of heterodimers of such molecules where the cysteine residues are retained (Moritz et al, 1995) and their excellent expression when I modified-to-remove these cysteines (Nolan et al, 1999) (FIG. 2). The efficacy of T cell functions through surface receptors are generally higher with higher surface expression, which the rescue (i.e., non-destruction) of heterodimers would allow. These chimeric molecules are introduced into patient T cells by gene therapy techniques, such as by retroviral vector transduction or other methods. This method of improving cell surface expression is cross-referenced (Junghans Provisional Patent No. 60/250,087).

In one example, IgTCR (FIG. 1) provides signal 1, which directs T cell killing (e.g., FIG. 5); IgCD28 (FIG. 7) provides signal 2, which suppresses activation induced cell death of T cells and allows sustained proliferation and survival (FIG. 6); and IgLFA1, which provides signal 3 and supports secretion of interleukin 2, an essential T cell growth factor. Combinations of signals can yield improved T cell survival and tumor cell killing (FIG. 6). The invention allows for use of these and/or analogous chimeric molecules of hMN14 alone or in any combination.

The combination use of such chimeric molecules in treatment of cancers is a further part of the claim. This applies an understanding that more than one signal is required for sustained antitumor efficacy. This application specifically envisions that the same antibody binding domain is applied in the additional chimeric receptor molecules such that encounter with the same tumor antigen successfully triggers more than one signal in the effector cell. Alternatively, additional signaling chimeric molecules may have engineered Ig specificities which direct them to different surface molecules on the tumor cell, rather than to the same one, to avoid binding site competition or to regulate the amount of receptor stimulation where this regulation enhances the desired outcome of antitumor efficacy in therapy.

The purpose of this invention is to educate immune effector cells to attack GD3-expressing or PSMA-expressing tumor cells. Advantages are that the sequences used to recognize GD3 or PSMA in their conjugation with T cell molecules leads to direct recognition of GD3+ or PSMA+ tumors by human T cells, and hinge and sFv linker modifications make the surface expression more efficient with advantages in anti-tumor activity. Presently, treatments for these cancers are chemotherapy, immunotherapy, surgery and radiation, which are rarely or never curative for metastatic disease. A critical component of this patent for therapy is the specific antibodies that recognize these antigens. No other IgTCR or Ig-T cell molecules has the amino acid sequence of the GD3 or PSMA antibody recognition domains specified herein, and which I have proven to be effective (e.g., FIG. 5). There is no patent of these sequences in chimeric state with T cell or other effector cell molecules, or with the use of a modified hinge structure. This purpose is expanded to other tumor-associated antigens as appropriate to other antibodies as obtained with rights by the inventor or his agents, which will be appended as supplemental claims at such appropriate time.

An invention exists as to the general chimeric Ig molecules with cell receptor proteins (Capon et al, 1995). This invention is distinguished by the uniqueness of the antibody sequences employed, by the new concept in modification of hinge domains that improves the expression in cells, and by the combination of such chimeric receptor molecules expressed in effector cells which are stimulated in concert specifically by the same tumor antigen or by a different tumor antigen or antigens. The sequences of these antibodies were not previously patented. The claims of the present patent are restricted to the use of these antibodies and sequences in the preparation of these chimeric molecules for the purposes herein described. These claims as pertaining to these sequences do not extend to other potential uses of these antibodies and their derivatives, which are reserved for potential future applications.

What is claimed is:

1. A chimeric molecule comprised of the GD3 binding domain of antibody MB3.6, with variable gene sequences as specified in FIGS. 4A-C, as a single chain antibody with a (GGSGS)3 linker, the zeta signaling chain of the T cell receptor and an intervening CD8α hinge in which the cysteine residues have been mutated.

2. A chimeric molecule comprised of the PSMA binding domain of antibody 3D8, with variable gene sequences as specified in FIGS. 4D&E, as a single chain antibody with a (GGSGS)3 linker, the zeta signaling chain of the T cell receptor and an intervening CD8α hinge in which the cysteine residues have been mutated.

3. A chimeric molecule comprised of the PSMA binding domain of antibody 4D4, with variable gene sequences as specified in FIGS. 4F&G, as a single chain antibody with a (GGSGS)3 linker, the zeta signaling chain of the T cell receptor and an intervening CD8α hinge in which the cysteine residues have been mutated.

4. A chimeric molecule comprised of the PSMA binding domain of antibody 3E11, with variable gene sequences as specified in FIGS. 4H&I, as a single chain antibody with a (GGSGS)3 linker, the zeta signaling chain of the T cell receptor and an intervening CD8α hinge in which the cysteine residues have been mutated.

5. Molecules of claim 1-4 in which other signaling chains of T cells or other cell types are substituted, or in which a different hinge molecule or no hinge molecule is substituted, or a combination thereof.

6. Molecules of claim 1-5 in which at least one of the CDRs of the heavy chain and one of the CDRs of the light chain are preserved in a form (e.g., sFv or Fab) that maintains the binding of the antigen, and/or in which the linker is of different composition. For MB3.6, this specification may be met by one CDR of the heavy chain to maintain antigen binding because of the small size of the ganglioside antigen.

7. Molecules of claim 1-6 which has been modified in DNA or protein sequence but which retains the specificity and action of these molecules.

8. The use of molecules of claims 1-7 expressed in T cells or NK cells or other effector cells to treat patients with cancers expressing the GD3 (MB3.6 derivatives) or PSMA antigen (3D8, 4D4, 3E11 derivatives).

9. The combination use of molecules of claims 1-7 expressed in T cells or NK cells or other effector cells to treat patients with cancers expressing the GD3 (MB3.6 derivatives) or PSMA antigen (3D8, 4D4, 3E11 derivatives), together with each other or with heterologous constructs to engage additional stimulatory and functional properties of the effector cells to enhance the antitumor therapeutic efficacy.


SEQUENCE LISTING (19 sequences)

SEQ ID NO: 1 - DNA, 7654 bp, Homo sapiens and Mus sp.
  CDS (2428)..(3759): Chimeric IgTCR sequence contained in retroviral vector.  Retroviral vector sequence (non-coding regions) are incidental to the invention.  The translated (coding region) is relevant to the invention. (pertinent to Figure 3.)
aagcttgcatgcctgcaggtcgactctaggcacataaagaaaaacataactaaccaagctgcagccgagacagtgaaaagaaccgttaaaacggtttgttttaaataaactgaattatttagagtcatttctttggtaggaaagtacattggcacgtaaaggagcccaaagcaatctgtggaaagcccaggctgggagcccagcagtttgcatcccctcctggcgtgtacctaagggtttcttaattgtgtggtttctaaatcttccagagggtttgtctcattcacttccacttcggtgcacaatacttggacgcggatttactgtcttagcatctatcggtggcccttcgattgaggctgaacctgaggcccacttcttcagcttgttaaggagagcacaagcaccagaagaggctgacccggcagacctgtgggcatttttaacaagggcctcctgggtctgtgggaggcaggcttacataaggtgcaaattagaaatataaataataagcccatatcaatttgtcatctttttttaagctcaagttttgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggcttctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgggatggagctgtatcatcctcttcttggtagcaacagctacaggtgtccactccgacatccagctgacccagagcccaagcagcctgagcgccagcgtgggtgacagagtgaccatcacctgtaaggccagtcaggatgtgggtacttctgtagcttggtaccagcagaagccaggtaaggctccaaagctgctgatctactggacatccacccggcacactggtgtgccaagcagattcagcggtagcggtagcggtaccgacttcaccttcaccatcagcagcctccagccagaggacatcgccacctactactgccagcaatatagcctctatcggtcgttcggccaagggaccaaggtggaaatcaaacgaggtggctcaggatcgggtggatccggctctggtggctcaggatcggaggtccaactggtggagagcggtggaggtgttgtgcaacctggccggtccctgcgcctgtcctgctccgcatctggcttcgatttcaccacatattggatgagttgggtgagacaggcacctggaaaaggtcttgagtggattggagaaattcatccagatagcagtacgattaactatgcgccgtctctaaaggatagatttacaatatcgcgagacaacgccaagaacacattgttcctgcaaatggacagcctgagacccgaagacaccggggtctatttttgtgcaagcctttacttcggcttcccctggtttgcttattggggccaagggaccccggtcaccgtctccagtgctaagcccaccacgacgccagcgccgcgaccaccaacaccggcgcccaccatcgcgtcgcagcccctgtccctgcgcccagaggcggctcggccagcggcggggggcgcagtgcacacgagggggctggacttcgccctggatcccaaactctgctacctgctggatggaatcctcttcatctatggtgtcattctcactgccttgttcctgagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaactcgacgcggccgcggatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctctctgagtgattgactacccgtcagcggggtctttcagtttctcccacctacacaggtctcactaacattcctgatgtgccgcagggactccgtcagcccggtttttgtttataataaaatgcaagaacagtgttcccttcaagccagactacatcctgactctcggctttataaaagaatgttgaagggctctgtggactatctgccacacgactttttaagatttttatgcctcctggatgagggatttagtcaatctatcctcgtctattttgctggcttctccgtattttaaatttctagtttgcactcccttcctgagagcacggcgattgcagagtagttaatactctgagggcaggcttctgtgaaaaggttgcctgggctcagtgtgagattttgccataaaaaggggtcctgcccctgtgtacagacagatcggaatctagagtgcatactcagagtccccgcggttccggggctctgatctcagggcatctttgcctagagatcctctacgccggacgcatcgtggccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggctcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 2 - protein, 443 aa, Homo sapiens and Mus sp.
MGWSCIILFLVATATGVHSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSAKPTTTPAPRPPTPAPTIASQPLSLRPEAARPAAGGAVHTRGLDFALDPKLCYLLDGILFIYGVILTALFLRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 504 bp, Mus sp.
  CDS (6)..(425): MB3.6 Heavy chain V region, plus leader
tcaccatgaacttcgggttcagcttgattttccttgtccttgttttaaaaggtgtccagtgtgaagtggtggtggtggagtctgggggaggcttcgtgaagcctggagggtccctgaaactctcctgtgcagccgctggattcactttcagtagatatgccatgtcttgggttcgccagactccggagaagaggctggagtgggtcgcaaccataagtagtggtggtagtcacacctactatccagacagtgtgaaggggcgattcaccatctccagagacaatgccaagaacaccctgtacctgcaaatgagcagtctgaggtctgaggacacggccatatattactgtgcaagaccgggttacgacaggggggcctggtttttcgatgtctggggcgcagggaccacggtcaccgtctcctcaggtaagtgtgtcagggtttcacaagagggactaaagacatgtcagctaatgtgtgactaatggtaatgtcactaagctt

SEQ ID NO: 4 - protein, 140 aa, Mus sp.
MNFGFSLIFLVLVLKGVQCEVVVVESGGGFVKPGGSLKLSCAAAGFTFSRYAMSWVRQTPEKRLEWVATISSGGSHTYYPDSVKGRFTISRDNAKNTLYLQMSSLRSEDTAIYYCARPGYDRGAWFFDVWGAGTTVTVSS

SEQ ID NO: 5 - DNA, 483 bp, Mus sp.
  CDS (16)..(399): MB3.6 Light chain V region, plus leader
agggaaagctcgaagatggttttcacacctcagatacttggacttatgcttttttggatttcagcctccagaggtgatattgtgctaactcagtctccagccaccctgtctgtgactccaggagatagcgtcagtctttcctgcagggccagccaaattattagcaacaacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacagagtaacagctggcctctcacgttcggctcggggacaaagctggagatcaaacggcgtaagtgtgtcagggtttcacaagagggactaaagacatgtcagctaatgtgtgactaatggtaatgtcacttgtcaggatcc

SEQ ID NO: 6 - protein, 128 aa, Mus sp.
MVFTPQILGLMLFWISASRGDIVLTQSPATLSVTPGDSVSLSCRASQIISNNLHWYQQKSHESPRLLIKYASQSISGIPSRFSGSGSGTDFTLSINSVETEDFGMYFCQQSNSWPLTFGSGTKLEIKR

SEQ ID NO: 7 - DNA, 258 bp, Mus sp.
  misc_feature (1)..(258): Light chain leader plus sFv of MB3.6
gatatcagatctcagctgtctagacatatggttttcacacctcagatannnnnnnnnnnnnnnnnnnnnnnnnnnnnnnnnnnnnnnnnnnnnnnnnnnnnnnnngggacaaagctggagatcaaaggtggctcaggatcgggtggagccggctctggtggctcaggatcggaagtggtggtggtggagnnnnnnnnnnnnnnnnnnnnnnnnnnnnnnnnnnnnnnnnnnnnnnnnaccacggtcaccgtctccagt

SEQ ID NO: 8 - DNA, 682 bp, Mus sp.
  CDS (20)..(418): 3D8 Heavy chain V region, plus leader
tgaacacggacccctcaccatgaacttcgggctcagcttgattttccttgtccttgttttaaaaggtgtccagtgtgaagtgaaggtggtggagtctgggggaggcttagtgaagcctggagcgtctctgaaactctcctgtgcagcctctggattcactttcagtaactatggcatgtcttgggttcgccagacttcagacaagaggctggagtgggtcgcatccattagtagtggtggtgatagcaccttctatgcagacaatgtaaagggccgattcaccatctccagagagaatgccaagaacaccctgtacctgcaaatgagtagtctgaagtctgaggacacggccttgtattactgtgcaagagacgatctatttaactggggccaaggcaccactctcacagtctcatcagccaaaacaacagccccatcggtctatccactggcccctgtgtgtggagatacaattggctcctcggtgactttaggatgcctggtcaagggttatttccttgagccagtgaccttgacctggaactctggatccctgtccagtggtgtgcacatcttcccagctgtcttgcagtctgacctctacaccctcagcagctcagtgactgtaacctcgagcacctggcccagccagtccatcacttgcaatgtggcccacccggca

SEQ ID NO: 9 - protein, 133 aa, Mus sp.
MNFGLSLIFLVLVLKGVQCEVKVVESGGGLVKPGASLKLSCAASGFTFSNYGMSWVRQTSDKRLEWVASISSGGDSTFYADNVKGRFTISRENAKNTLYLQMSSLKSEDTALYYCARDDLFNWGQGTTLTVSS

SEQ ID NO: 10 - DNA, 729 bp, Mus sp.
  CDS (15)..(410): 3D8 Light chain V region, plus leader
ccgttgccgtcgtgatgagtcctgcccagttcctgtttctgttagtgctctggattcaggaaaccaacggtgatgttgtaatgacccagactccactcactttgtcggttaccattggacaaccagcctctatctcttgcaagtcaagtcagagcctcttatatagtaatggaaaaacctatttgaattggttattacagaggccaggccagtctccaaagcgcctaatctatctggtgtctaaactggactctggagtccctgacaggttcactggcagtggatcaggaacagattttacactgaaaatcagcagagtggaggctgaggatttgggagtttattactgcgtgcaaggtacacattttcctcacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtt

SEQ ID NO: 11 - protein, 132 aa, Mus sp.
MSPAQFLFLLVLWIQETNGDVVMTQTPLTLSVTIGQPASISCKSSQSLLYSNGKTYLNWLLQRPGQSPKRLIYLVSKLDSGVPDRFTGSGSGTDFTLKISRVEAEDLGVYYCVQGTHFPHTFGGGTKLEIKR

SEQ ID NO: 12 - DNA, 736 bp, Mus sp.
  CDS (14)..(430): 4D4 Heavy chain V region, plus leader
actgactctaaccatgggatggagatggatctttcttttcctcctgtcaggaactgcaggtgtccattgccaggttcagctgcagcagtctggacctgagctggtgaagcctggggctttagtgaagatatcctgcaaggcttctggttacaccttcacaagctacgatataaactgggtgaagcagaggcctggacagggacttgagtggattggatggatttatcctggagatggtggtactaattacaatgagaaattcaagggcaaggccacactgactgcagacaaatcctccagcacagcctacatgcagctcagtagcctgacttctgagaactctgcagtctatttctgtgcaagagggggtaacttcccttcttatgctatggactactggggtcaaggaacctcagtcaccgtctcctcagccaaaacgacacccccatctgtctatccactggcccctggatctgctgcccaaactaactccatggtgaccccgggatgcctggtcaagggctatttccctgagccagtgacagtgacctggaactctggatccctgtccagcggtgtgcacaccttcccagctgtcctgcagtctgacctctacactctgagcagctcagtgactgtcccctccagcacctggcccagcgagaccgtcacctgcaacgttgcccacccggccagcagcaccaaggtggacaagaaaattgtgcccagggattgt

SEQ ID NO: 13 - protein, 139 aa, Mus sp.
MGWRWIFLFLLSGTAGVHCQVQLQQSGPELVKPGALVKISCKASGYTFTSYDINWVKQRPGQGLEWIGWIYPGDGGTNYNEKFKGKATLTADKSSSTAYMQLSSLTSENSAVYFCARGGNFPSYAMDYWGQGTSVTVSS

SEQ ID NO: 14 - DNA, 504 bp, Mus sp.
  CDS (7)..(402): 4D4 Light chain V region, plus leader
ctcaaaatgaagttgcctgttaggctgttggtgctgatgttctggattcctgcttccaacagtgatgttttgatgacccaatctccactctccctgcctgtcagtcttggagatcaagcctccatctcttgcagatctagtcagagcattgtccatagtaatggagacacctatttagaatggtacctgcagaaaccaggccagtctccaaagctcctgatctacaaggtttccgaccgattttctggggtcccagacaggttcagtggcagtggatcagggacagatttcacactcaagatcagcagagtggaggctgaggatctgggagtttatttctgctttcaaggttcacatgttccgtacgcgttcggaggggggaccaagctggaaataaaacgggctgatgctgcaccaactgtatccatcttcccaccatccagtgagcagttaacatctggaggtgcctcagtcgtgtgcttcttgaacaacttctaccccaaa

SEQ ID NO: 15 - protein, 132 aa, Mus sp.
MKLPVRLLVLMFWIPASNSDVLMTQSPLSLPVSLGDQASISCRSSQSIVHSNGDTYLEWYLQKPGQSPKLLIYKVSDRFSGVPDRFSGSGSGTDFTLKISRVEAEDLGVYFCFQGSHVPYAFGGGTKLEIKR

SEQ ID NO: 16 - DNA, 761 bp, Mus sp.
  CDS (62)..(478): 3E11 Heavy chain V region, plus leader
cctggattcaatttccagttcctcacattcagtgatcagcactgaacacggacccctcaccatgaacttcgggctcagcttgattttccttgtccttgttttaaaaggtgtccagtgtgaagtgaaactggtggagtctgggggagacttaatgaaccctggagcgtctctgaaactctcctgtgcagcctctggattcagtttcagtaactatggcatgtcttgggttcgccagacttcagacaagaggctggagtgggtcgcttccattagtacgggtggtgctaataccttctatccagacaatgtaaagggccgattcaccatttccagagagaatgccaagaacaccctatacctgcaaatgagtagtctgaagtctgaggacacggccttgtatttctgtgcaagagatagtcactccgtaggttgttggtttgctacctggggccaagggactctggtcactgtctctgcagccaaaacaacacccccatcagtctatccactggcccctgggtgtggagatactactggttcctccgtgactctgggatgcctggtcaagggctacttccctgagtcagtgactgtgacttggaactccggatccctgcccagcagtgtgcacaccttcccagctctcctgcagtctggactctacactatgagcagctcagtgactgtcccctccagcacctggccaagccagaccgttacctgcagtgttgctcacccagccagcagcaccacggtggaca

SEQ ID NO: 17 - protein, 139 aa, Mus sp.
MNFGLSLIFLVLVLKGVQCEVKLVESGGDLMNPGASLKLSCAASGFSFSNYGMSWVRQTSDKRLEWVASISTGGANTFYPDNVKGRFTISRENAKNTLYLQMSSLKSEDTALYFCARDSHSVGCWFATWGQGTLVTVSA

SEQ ID NO: 18 - DNA, 698 bp, Mus sp.
  CDS (6)..(401): 3E11 Light chain V region, plus leader
ccagcatgggcatcaagatggaatcacagactctggtcttcatatccatactgctctggttatatggagctgatgggaacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccatcgtcaag

SEQ ID NO: 19 - protein, 132 aa, Mus sp.
MGIKMESQTLVFISILLWLYGADGNIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR